(19) United States
(12) Patent Application Publication
(10) Pub. No.: US 2015/0133416 A1
(43) Pub. Date: May 14, 2015

(54) Pharmaceutical Composition Comprising Abiraterone Acetate

(71) Applicant: Jagotec AG, Muttenz (CH)

(72) Inventors: Pascal Grenier, Kappelen (FR); Guy Vergnault, Kembes (FR)

(21) Appl. No.: 14/398,841

(22) PCT Filed: May 3, 2013
(86) PCT No.: PCT/EP2013/059307

§ 371 Date: Nov. 4, 2014

(30) Foreign Application Priority Data
May 4, 2012 (GB) 1207886.1

(51) Int. Cl. A61K 31/58; A61K 47/12; A61K 47/26; A61K 47/22; A61K 47/20

(52) U.S. Cl. CPC A61K 31/58; A61K 47/22; A61K 47/20; A61K 47/26; A61K 47/12
USPC 514/176

(57) ABSTRACT

Pharmaceutical composition comprising abiraterone acetate, which exhibits a reduced food effect and improved bioavailability.

The invention relates to a pharmaceutical composition comprising abiraterone or a pharmaceutically acceptable salt thereof. The invention is further concerned with methods of treating certain cancers in mammalian subjects using said composition.

Prostate cancer is the most common cancer in males, generally occurring later in life. It is a slow-growing cancer and early detection can result in a surgical cure if the cancer is confined to the prostate gland. Surgical treatment involves complete removal of the prostate in a procedure called radical prostatectomy. Patients at this stage of the disease also have other options including radiation, cryotherapy or just watchful waiting.

If the cancer has spread beyond the prostate gland, surgery is not an option and patients are put on hormonal therapy, a chemical castration which blocks most of the production of testosterone. The hormone testosterone spurs the growth of both normal and cancerous prostate cells. By sharply ablating the level of testosterone, hormonal therapy can generally control the disease for around 2-3 years before it continues to progress.

After hormonal therapy fails, the next and last course of action has been chemotherapy treatment. Taxotere offers a means of treatment for this stage of the disease but it is a difficult drug to tolerate. Provenge, a cancer vaccine, was approved in May of 2010 for use after hormonal therapy has failed and before chemotherapy is started, creating an opportunity for patients to obtain a therapeutic benefit before chemotherapy.

However, cancer cells that adapt to reduced testosterone levels achieved by androgen deprivation treatment will continue grow. In April 2011, the FDA approved a new product indicated for the treatment of patients with metastatic castration-resistant prostate cancer (CRPC) who have received prior chemotherapy containing docetaxel. Abiraterone's mechanism of action results in further blockade of testosterone production resulting in reduced tumour growth and survival when all other currently used drugs have failed.

Abiraterone is an irreversible inhibitor of CYP17 (17^{a}-hydroxylase/C17,20-lyase enzyme), an androgen biosynthesis inhibitor. This enzyme is expressed in testicular, adrenal and prostatic tumor tissues. Inhibition of this enzyme results in further reduction of testosterone synthesis in patients already undergoing androgen deprivation therapy (e.g. chemical and surgical castration).

Abiraterone, used in combination with prednisone, is indicated for the treatment of castration resistant prostate cancer (CRPC) patients.

Abiraterone acetate is the acetyl ester of abiraterone. It is the active ingredient in the commercially available tablet ZYTIGA®. ZYTIGA® comprises abiraterone acetate and the excipients lactose monohydrate, microcrystalline cellulose, croscarmellose sodium, povidone (K29/K32), sodium laurilsulfate, magnesium stearate and colloidal anhydrous silica (EU marketing authorisation no. EU/1/11/714/001). Each ZYTIGA® tablet weighs 715 mg and contains 250 mg of abiraterone acetate, 189 mg of lactose and 6.8 mg of sodium. The recommended dose is 1000 mg abiraterone acetate QD, in combination with prednisone 5 mg bid.

A problem exists with abiraterone in that its pharmacokinetic properties are affected by the prandial status of a patient receiving treatment, i.e. it exhibits a ‘food effect’. In particular, the bioavailability of abiraterone increases with food. Thus, after a low fat meal, Cmax and AUC are elevated 7- and 5-fold compared with the fasted state, whereas after a high fat meal there is a 17- and 10-fold elevation. As such, patients receive instructions to take ZYTIGA on an empty stomach, that is, no food intake during a period of 2 hours before dosing, and no food for a period one hour after dosing. This can lead to both inter- and intra-subject variability in bioavailability of this drug.

ZYTIGA® is administered in a fasted state in an attempt to minimise the erratic and unpredictable bioavailability demonstrated in the fed state. However, ZYTIGA® has poor bioavailability in fasted subjects and must therefore be administered at a very high daily dose.

Administration of a drug composition with food may change its bioavailability by affecting either the drug substance or the composition in which the drug substance is formulated. In practice, it is difficult to determine the exact mechanism by which food changes the bioavailability of a drug substance or composition. Food can alter the bioavailability of a drug by different mechanisms, including: delayed gastric emptying; stimulation of bile flow; changed gastrointestinal pH; increased visceral blood flow; changed luminal metabolism; and physical or chemical interactions of food components with the drug compound or one or more excipients contained in the composition. Still further, food-induced changes in gut physiology can contribute to these effects, or there may be instability of the drug substance.

Food can affect the uptake of a drug in many ways. It can affect the maximum plasma concentration (Cmax) but not affect the extent of uptake of the drug. On the other hand, it might affect time to maximum plasma concentration (Tmax) but not Cmax. By way of example, levetiracetam is known to have Cmax and Tmax that are both affected by food but its overall uptake is unaffected. For some drugs, the bioavailability is enhanced by food (positive food effect), while in others the bioavailability is reduced (negative food effect). Food may create fed state conditions that assist in better solubilisation of a drug, but may also create a certain number of physiological changes that improve intestinal membrane permeation (e.g. the formation of mixed micelles).

Food effects have been observed with a diversity of drug substances, formulated in a variety of ways. For example, benzimidazole proton pump inhibitor products are affected by the presence of food in the stomach at the time of dosing or shortly thereafter: Omeprazole 40 mg delayed release capsules show no food effect with apple sauce, but omeprazole 20 mg delayed release capsules exhibits a 25% reduction in Cmax when administered with apple sauce. Delayed release esomeprazole magnesium capsules exhibit a decrease in AUC when administration occurs after food intake; the rabeprazole sodium product gives a delay in Tmax when administered with a high fat meal; delayed release pantoprazole sodium has a highly variable Tmax, which can increase significantly when given with food; and delayed release lansoprazole formulations exhibit both diminished Cmax and AUC if the drug is given shortly after food.

Accordingly, whereas it is not uncommon for the pharmacokinetic properties of a drug substance to be affected by the presence or absence of food in the stomach when administered to a patient, or before the drug substance has passed from the stomach, the presence of a food effect, its direction (positive or negative) and its magnitude is unpredictable. A particular formulation strategy that reduces or eliminates a food effect with respect to one particular drug substance or composition might not necessarily have a similar effect with other drug substances or compositions.

Abiraterone acetate is classified as Class IV compound according to the Biopharmaceutical Classification System (BCS)^{1/}. BCS Class IV drugs typically have poor bioavailability and are characterized by low solubility and low permeability. That is, their bioavailability is limited by both solvation rate and permeability. Usually they are not well absorbed over the intestinal mucosa and can exhibit a high degree of absorption variation, which makes them particularly difficult to administer orally. Abiraterone has a very high log P and exhibits poor aqueous solubility and poor mucous membrane permeability. These factors alone or in combination may contribute to the observed large food effect and intra-subject and inter-subject variability of this drug substance in its current commercial presentation. However, abiraterone poses particular formulation problems, not only because of its erratic bioavailability, but also because of its poor bioavailability in fasted subjects, it must be administered at such a high daily dose (1000 mg/day). See e.g. BSC guidance published by the FDA entitled “Waiver of in vivo bioavailability and bioequivalence studies for immediate-release solid oral dosage forms based on a Biopharmaceutics Classification System”, August 2000; and http://www.fda.gov/AboutFDA/CentersOffices/OfficeofMedicalProductsandTobacco/CDER/ucm128219.htm

Furthermore, despite the fact that it is manufactured exclusively in a single form (Form A), and that the particle size is fine and its distribution narrow (D50 comprised between 3 and 10 microns), bioavailability is not improved nor is the food affect addressed.

There remains a need to develop formulations, which can improve the bioavailability of abiraterone in fasting conditions, reduce the food effect in the fed state as well as the high intra- and inter-subject variability that is observed with this substance in its current commercial presentation, i.e. ZYTIGA®.

The invention addresses shortcomings of abiraterone in its current commercial presentation in order to provide better tolerated and safer dosage forms that achieve more predictable and consistent plasma exposure, which may allow a lowering of dosage strength and total daily dose. For example, the invention provides improved abiraterone bioavailability in fasting conditions and reduces the food effect of abiraterone in the fed state.

The invention provides in a first aspect a pharmaceutical composition comprising abiraterone acetate dissolved or dispersed in a pharmaceutically acceptable carrier, which composition when administered in a therapeutically effective amount, for example when the composition comprises 1000 mg, 900 mg, 800 mg, 750 mg, 600 mg, 500 mg, 400 mg, 300 mg or 250 mg of abiraterone acetate, in a single dose orally to a mammalian subject in the fasted state exhibits improved bioavailability compared to the equivalent amount of abiraterone acetate in an orally administered single dosage form of the drug product that is commercially marketed under the trade name ZYTIGA®.

In particular, the invention provides a pharmaceutical composition comprising abiraterone acetate dissolved or dispersed in a pharmaceutically acceptable carrier, which composition when administered in a 250 mg single dose orally to a mammalian subject in the fasted state exhibits improved bioavailability compared to a 250 mg orally administered single dosage form of the drug product that is commercially marketed under the trade name ZYTIGA®.

In another aspect of the present invention there is provided a pharmaceutical composition as hereinabove defined, wherein said dosage fon 1, when administered to a mammalian subject in the fasted state is bioequivalent to the same dosage form administered to a mammalian subject in the fed state.

As used herein, the term “bioavailability” denotes the fraction of a drug substance that reaches systemic circulation after administration orally to a mammalian subject.

Parameters often used in the measurement of bioavailability are Tmax, Cmax and AUC₀₋ₜ, which are well known in the art and which are more fully explained in guidelines given by the US Food and Drug Administration (US FDA) and European Medicines Agency (EMEA).

“Tmax” denotes the time to reach the maximal plasma concentration (Cmax) after administration; whereas AUC 0-t denotes the area under the plasma concentration versus time curve from time 0 to time t, for example, AUC 0-24 is the area under the plasma concentration versus time curve from time 0 to time 24 hours.

The term “improved bioavailability” is intended to mean that administration of a composition according to the invention under fasted conditions will result in a bioavailability that is greater than the bioavailability obtained after administration of the same dosage strength of the commercially available product ZYTIGA®.

In another aspect of the invention there is provided a pharmaceutical composition comprising abiraterone acetate dissolved or dispersed in a pharmaceutically acceptable carrier, which composition when administered to a mammalian subject orally in fasting conditions in a therapeutically effective amount, for example in a single dose comprising 250 mg, 300 mg, 400 mg, 500 mg, 600 mg, 750 mg, 800 mg, 900 mg or 1000 mg arbiraterone acetate per day, exhibits a pharmacokinetic profile characterised by an AUC_{0-inf} that is higher than ZYTIGA® comprising the same dose of arbiraterone acetate in the fasted state.

In particular, there is provided a pharmaceutical composition comprising abiraterone acetate dissolved or dispersed in a pharmaceutically acceptable carrier, which composition when administered to a mammalian subject orally in fasting conditions at a single dose of 1000 mg per day exhibits a pharmacokinetic profile characterised by an AUC_{0-inf} that is higher than ZYTIGA® in the fasted state, more particularly an AUC_{0-inf} greater than 421 nM/1.ml.

In another aspect of the invention there is provided a pharmaceutical composition comprising abiraterone acetate dissolved or dispersed in a pharmaceutically acceptable carrier, which composition when administered to a mammalian subject orally in fasting conditions in a therapeutically effective amount, for example at a single dose comprising 250 mg, 300 mg, 400 mg, 500 mg, 600 mg, 750 mg, 800 mg, 900 mg or 1000 mg of arbiraterone acetate, exhibits a pharmacokinetic profile characterised by a Cmax that is higher than ZYTIGA® comprising the same dose of arbiraterone acetate in the fasted state.

In particular, there is provided a pharmaceutical composition comprising abiraterone acetate dissolved or dispersed in a pharmaceutically acceptable carrier, which composition when administered to a mammalian subject orally in fasting conditions at a single dose of 1000 mg exhibits a pharmacokinetic profile characterised by a Cmax that is higher than ZYTIGA® in the fasted state, more particularly a Cmax of about 70 ng/ml (at steady state for 1000 mg daily).

As used herein, the terms “bioequivalent”, “bioequivalence” or “bioequivalency” denote a scientific basis on which drug products are compared with one another. It is an important concept relating to the regulatory acceptance drug products, in particular generic drug products, in the sense that for generic approval, a generic drug should be bioequivalent to a reference drug product.

Drugs are bioequivalent if they enter circulation at the same rate and to the same extent when given in similar doses under similar conditions. Parameters often used in bioequivalence studies are Tmax, Cmax and AUC₀₋ₜ as defined above.

Bioequivalency of two compositions is established by a 90% Confidence Interval (CI) of between 0.80 and 1.25 for both Cmax and AUC under United States FDA regulatory guidelines, or a 90% CI for AUC of between 0.80 to 1.25 and a 90% CI for Cmax of between 0.70 to 1.43 under the European regulatory guidelines (EMA). The difference between a bioequivalence parameter described above measured after oral administration to a mammal should be less than about 125% and more than about 80% with respect to AUC and less than about 143% and more than about 70% for Cmax.

At least one of these parameters may be applied in order to determine whether or not bioequivalence is established. More particularly, AUC and Cmax are examined in order to determine whether or not bioequivalence is established.

The term “confidence interval” refers to a statistical range with a specified probability that a given parameter lies within the range.

Confidence intervals represent a reasoned statement about the true mean of a population based on a random sample. When taking a mean value from a random sample, most likely the mean of that sample will not be the true mean of the population, but only an estimate. The confidence interval represents a range of values around the sample mean that will include the true mean.

Confidence intervals are expressed as a percentage (usually 90 or 95%). This simply means that if a researcher was to take 100 random samples, he could be certain that 90 or 95 times that the range of values expressed by the confidence interval procedure would include the true mean of a population from which the samples were drawn.

In the context of the present invention, with regard to comparisons of bioavailability, a composition of the present invention may be compared with a reference composition that is the commercially available ZYTIGA® drug product. The ZYTIGA® products are tablets of abiraterone acetate developed by Jansen Biotech and approved by the FDA on Apr. 28, 2011 (application no. 202379).

The ZYTIGA® formulation includes the excipient sodium lauryl sulphate, which functions as a wetting agent and to help solubilisation. However, as abiraterone is a Class IV BCS drug, even once the drug is soluble it is then difficult to absorb due to its low permeability properties.

The pharmaceutically acceptable carrier employed in compositions of the present invention may comprise one or more lipid excipients selected from the group consisting of fatty acid esters, phospholipids, phosphatidyl compounds, glycosylceramides, fatty acids, nonionic surfactants, vitamin E tocopheryl succinate polyethylene glycol, glycerides, derivatives thereof, and mixtures thereof.

The majority of these lipid excipients also have surfactant characteristics and may function to improve both the solubility and permeability of abiraterone. The pharmaceutically acceptable carrier may further comprise additional surfactants and/or co-surfactants. Such excipients and surfactants/co-surfactants can assist in the dispersion and/or solubilisation of the drug substance, or facilitate emulsification of the drug substance upon contact with gastro-intestinal fluids, or maintain it in a solubilised form during gastro-intestinal transit thereby increasing absorption.

By addressing the food effect problem, pharmaceutical compositions of the present invention may allow patients more freedom to choose when to administer their medication. The absence or reduction of a food effect can be concluded when the 90% confidence intervals for the ratio of the geometric means based on log transformed data in clinical studies of fed and fasted treatments fall within 80% to 125% for AUC and 70% to 143% for Cmax. The difference between a bioequivalence parameter measured after oral administration to a mammal with and without food, respectively, is more than about 80% and less than 125% for AUC and more than about 70% and less than 143% for Cmax.

In another aspect of the present invention there is provided a pharmaceutical composition comprising abiraterone acetate dissolved or dispersed in a carrier material, as hereinabove described, wherein the difference between a bioequivalence parameter measured after oral administration to a mammalian subject with and without food, respectively, is more than about 80% and less than 125% with respect to AUC and more than about 70% and less than 143% for Cmax.

As stated above, the comparison for determining the presence or absence of a food effect is made between a population of fed and fasted patients. As used herein, a patient in a fed state is defined as having been fed a high fat meal, especially when fed at least 1000 calories, 50% of which are from fat as more fully described in United States FDA or EMEA Guidelines. Alternatively, the fed patient may have taken a medium or light fat meal comprising 30% fat as set forth in AHA (American Heart Association) guidelines.

Furthermore, a fed patient may be defined as somebody who has fasted, e.g. for at least 10 hours overnight and then has consumed an entire test meal within 30 minutes of first ingestion. In a representative method, the pharmaceutical composition is administered with 180 ml of water within 5 minutes after completion of the meal. No food is then allowed for at least 4 hours post-dose. Water can be allowed ad libitum after 2 hours. A representative high fat high calorie test meal comprises 2 eggs fried in butter, 2 strips of bacon, 2 slices of toast with butter, 4 ounces of hash brown potatoes, and 8 ounces of whole milk to provide 150 protein calories, 250 carbohydrate calories, and 500 to 600 fat calories.

A patient in a fasted state may be defined as a subject who has not eaten any food, e.g. the patient has fasted for at least 10 hours before the administration of a pharmaceutical composition and who does not eat any food and continues to fast for at least 4 hours after the administration. The pharmaceutical composition is preferably administered with 180 ml of water during the fasting period, and water can be allowed ad libitum after 2 hours.

In another aspect of the invention there is provided a method of reducing or eliminating the difference between one or more bioequivalence parameters (defined above) measured after oral administration of a pharmaceutical composition defined herein to a mammalian subject in a fasted state compared with a mammalian subject in a fed state, the method comprising the step of administering to patients a pharmaceutical composition as hereinabove defined.

More particularly, in said method, the difference in said bioequivalence parameter(s) can be measured in accordance with the bioequivalence guidelines given by the U.S. Food and Drug Administration or the corresponding European regulatory agency (EMA).

In a particular embodiment of the method set forth above, one or more of the pharmacokinetic parameters AUC or Cmax of said pharmaceutical composition administered orally to a fasted patient is increased relative to said parameter(s) when said composition is administered orally to a patient in a fed state.

The pharmaceutically acceptable carrier employed in compositions of the present invention may comprise one or more lipid excipients or surfactants and/or co-surfactants. Such excipients and surfactants/co-surfactants can assist in the dispersion and/or solubilisation of the drug substance, or facilitate emulsification of the drug substance upon contact with gastro-intestinal fluids, or maintain it in a solubilised form during gastro-intestinal transit thereby increasing absorption.

Said one or more lipid excipients may be selected from the group consisting of fatty acid esters, phospholipids, phosphatidyl compounds, glycosylceramides, fatty acids, nonionic surfactants, vitamin E tocopheryl succinate polyethylene glycol, glycerides, derivatives thereof, and mixtures thereof.

A particular class of lipid excipient that may be used in the present invention is the class of polyethylene glycol glycerides. They are typically comprised of mono-, di- and tri-glycerides and mono- and di-esters of polyethylene glycol. These materials are commonly available under the trademark GELUCIR®. Particularly suitable GELUCIRE® materials are those having a high HLB (e.g. HLB of about 10 to 18), more particularly GELUCIRE® 44/14 (lauroyl macrogol-32 glycerides). GELUCIRE® is a lipid excipient that also has surfactant properties and can be employed to form a self-emulsifying formulation or a self microemulsifying formulation. GELUCIRe 44/14 comprises a well-defined mixture of mono-, di-, and tri-glycerides and mono- and di-fatty acid esters of polyethylene glycol. It is synthesized by an alcoholysis/esterification reaction using hydrogenated palm kernel oil and PEG1500 as starting materials. The predominant fatty acid is lauric acid (C₁₂). It has a melting point of 42-46° C. and an HLB of 14. It is also known as lauroyl macrogol-32 glycerides.

The pharmaceutically acceptable carrier can be employed to form liquid, semi-solid or powder formulations that can be filled into capsules or can be admixed with other excipients to form powders for tabletting, as will be more fully described hereinbelow.

As used herein, “HLB” means “hydrophilic-lipophilic balance,” which is an empirical quantity, on an arbitrary scale, that is a measure of the polarity of a surfactant or mixture of surfactants.

Lipid excipients can include sucrose fatty acid esters, such as sucrose stearate, sucrose palmitate, sucrose laurate, sucrose behenate, sucrose oleate, sucrose erucate, and the like, and mixtures thereof; phospholipid derivatives, phosphatidyl derivatives, glycosylceramides derivatives, fatty acid derivatives, nonionic surfactants, vitamin E tocopheryl succinate polyethylene glycol (TPGS) derivatives, Gelucir® series surfactants, glyceride derivatives, and the like, and mixtures thereof.

Phospholipids can include phosphoglycerides. Glycerides include mono-, di-, and tri-esters of glycerol. Fatty acids are generally straight-chain carboxylic acid compounds ranging from three to 18 carbons. Fatty acids include saturated compounds, such as butyric, caproic, caprylic, capric, lauric, myristic, palmitic, and stearic acids, and unsaturated acids containing one or more double bonds per molecule. The most common of the unsaturated fatty acids are oleic acid, linoleic acid, and linolenic acid. Ceramides are N-acyl fatty acid derivatives of a sphingosine, thus, glycosylceramides are ceramides bonded to saccharide radicals.

Phosphatides according to the present invention can include phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), distearoylphosphatidylcholine (DSPC), diarachidonyiphosphatidyicholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidyl glycerols, including distearoylphosphatidylglycerol (DSPG); phosphatidylinositol; phosphatidic acids, including dipalmitoylphosphatidic acid (DPPA) and distearoylphosphatidic acid (DSPA); and the like; and mixtures thereof.

Fatty acids can include palmitic acid, stearic acid, oleic acid, arachidonic acid, and the like, and mixtures thereof.

Surfactants can include polyoxyethylene-polyoxypropylene glycol block copolymers, sorbitan fatty acid esters, and fluorine-containing surfactants. Illustrative of the polyoxyethylene-polyoxypropylene glycol block copolymers are α-hydroxy-ω-hydroxypoly(oxyethylene)-poly(oxypropylene)-poly(oxyethylene) block copolymers. These latter block copolymers are generally referred to as poloxamer copolymers. Examples of poloxamer copolymers that are particularly suitable for use in the present invention include, for example, poloxamer F68, poloxamer L61, and poloxamer L64. These poloxamer copolymers are commercially available from Spectrum 1100 (Houston, Tex.).

Sorbitan fatty acid esters include, for example, poly(oxy-1,2,-ethanediyl) derivatives of higher alkyl esters of sorbitan. Examples of such esters of sorbitan include, for example, sorbitan monolaurate, sorbitan monooleate, sorbitan monopalmitate, and sorbitan monostearate. These, as well as other derivatives of sorbitan, are typically referred to as polysorbates, including, for example, polysorbate 20, polysorbate 40, polysorbate 60, and polysorbate 80. Various of the polysorbates are commercially available from Spectrum 1100 (Houston, Tex.).

In a particular embodiment of the present invention the carrier contains a phospholipid- or ionic surfactant. The use of a phospholipid or ionic surfactant not only can act as an aid to drug solubilisation, it may also generate an environment around the drug substance that mimics the environment created by the release of endogenous or natural surfactants such as phospholipids as part of the process of digestion after the intake of food. It is believed that phospholipids or ionic surfactants may interact with the drug substance to foini micelles that in turn interact with the unstirred water layer (UWL)—a bicarbonate-rich layer of mucus that maintains pH levels at around 7 at the villi surface—and the intestinal mucosa, to enhance absorption therethrough.

The phospholipid useful in a composition according to the present invention may be a single phospholipid or a mixture of two or more phospholipids, for example a mixture of two or a mixture of three or a mixture of four or a mixture of five or a mixture of from six to about ten phospholipids. Phospholipids could be either from egg or soybean origin. Grades of phospholipids are mainly characterized by their content of phosphatidylcholine, lysophosphatidylcholine and phosphatidylethanolamine. For the present invention the preferred grade contains more than 70% of phosphatidylcholine.

Suitable phospholipids include saturated phospholipids; unsaturated phospholipids, naturally derived phospholipids, synthetic phospholipids and semi synthetic phospholipids, animal and plant phospholipids, egg phospholipids, soya bean phospholipids, corn phospholipids, wheat germ, flax, cotton, and sunflower seed phospholipids, milk fat phospholipids, purified phospholipids from these and other natural sources, glycerophospholipids, phosphatides, phospholipids containing fatty acid esters including palmitate, stearate, oleate, linoleate, and arachidonate, which esters can be mixtures and mixtures of isomers in the phospholipids, phospholipids composed of fatty acids containing one or more double bonds such as dioleoyl phosphatidylcholine and egg phosphatidylcholine that are not stable as powders but are hygroscopic and can absorb moisture and become gummy, phospholipids composed of saturated fatty acids that are stable as powders and are relatively less amenable to absorption of moisture, phosphatidyiserines, phosphatidylcholines, phosphatidylethanolamines, phosphatidylinositols, phosphatidylglycerols such as L-alpha-dimyristoyl phosphatidylglycerol also known as 1,2-dimyristoyl-sn-glycero-3-phospho (rac-1-1Jlycerol) and also known as DMPG: phosphatidic acid, hydrogenated natural phospholipids, and commercially available saturated and unsaturated phospholipids such as those available from Avanti Polar Lipids. Inc. of Alabaster, Ala., USA.

The phospholipid may be salted or desalted, hydrogenated, or partially hydrogenated. The phospholipid can be a mixture of these phospholipids.

Preferred phospholipids include Lipoid E80, Lipoid EPC, Lipoid SPC, DMPG, Phospholipon 100H, a hydrogenated soybean phosphatidylcholine, Phospholipon 90H, Lipoid SPC-3, egg phospholipid, purified egg phospholipid, and mixtures thereof. A preferred phospholipid is Lipoid E80 for egg source and Lipoid S75 for soya origin. The concentration of phospholipid added to the compositions prepared according to this invention can be present in the range of 0.1 to 50%, preferably 0.2 to 20%, and more preferably 0.4 to 15%. A preferred level of phospholipids, e.g. Lipoid E80 is from about 0.4% to 15%, more preferably from about 0.5% to about 10%, and most preferably from 2 to 5% of the drug amount. The amount of phospholipid mentioned is calculated on the basis of the drug free base.

Ionic surfactant useful in compositions of the present invention may comprise one or more anionic, cationic and zwitterionic surfactant materials.

A non-exhaustive list of anionic surfactants includes ammonium lauryl sulphate, dioctyl sodium sulfosuccinate, perfluorobutanesulfonic acid, perfluorononanoic acid, perfluorooctanesulfonic acid, perfluorooctanoic acid, sodium laureth sulphate, sodium lauryl sulphate, sodium octyl sulphate, sodium dodecylbenzenesulfonate, sodium lauroyl sarcosinate, sodium palmate, sodium stearate and triethanolamine lauryl sulphate.

A non-exhaustive list of cationic surfactants includes benzalkonium chloride, benzethonium chloride, cetrimonium bromide, cetrimonium chloride, dimethyldioctadecylammonium chloride, lauryl methyl gluceth-10 hydroxypropyl dimonium chloride, stearalkonium chloride, and tetramethylammonium hydroxide.

A non-exhaustive list of zwitterionic surfactants includes those based on primary, secondary, tertiary or quaternary ammonium surfactants.

Of course, the skilled person will appreciate that other ionic surfactants suitable for pharmaceutical use may be employed in the present invention.

The present invention provides in another of its aspects a composition as herein described, which releases abiraterone acetate according to the profile of an immediate release formulation. In a particular aspect of the invention, pharmaceutical compositions defined herein, when tested in a phosphate buffer pH 4.5 in USP apparatus II at the paddles speed of 50 rpm release at least 80% of their content within 60 minutes.

In another aspect of the invention there is provided a method of treating any of the conditions for which the product ZYTIGA® is indicated including Castration Resistant Prostate Cancer the method comprising the step of administering to a patient in need thereof a pharmaceutical composition as herein defined.

Abiraterone acetate in combination with prednisone is indicated for the treatment of patients with metastatic castration-resistant prostate cancer (CRPC) who have received prior chemotherapy. Abiraterone acetate may also be employed in a method of treating pre-chemotherapy CRPC and metastatic CRPC in combination with dutasteride. It may also be employed in the treatment of breast cancer, particularly metastatic breast cancer, including estrogen-resistant and metastatic breast cancer.

The recommended dosage of ZYTIGA® is 1000 mg (based on its salt, abiraterone acetate) once daily in adults, although patients with moderate baseline hepatic impairment should be administered only 250 mg once daily. ZYTIGA® must be taken on an empty stomach. No food should be consumed for at least two hours before the dose of ZYTIGA is taken and for at least one hour after the dose of ZYTIGA® is taken.

In yet another aspect of the present invention there is provided a process for preparing a pharmaceutical composition as defined herein, the process comprising the step of dispersing or dissolving abiraterone acetate in a suitable pharmaceutical carrier comprising one or more lipid excipients or surfactants and/or co-surfactants, and optionally one or more pharmaceutically acceptable excipients.

Details of one or more embodiments of the invention are set forth in the description below. Other features, objects and advantages of the invention will be apparent from the following description.

The pharmaceutical carrier may contain other excipients, the nature of which will depend upon the intended physical form of the pharmaceutical composition, i.e. whether the composition will be in the form of a compressed tablet, a capsule, a powder or the like. Generally however, excipients will be employed that are useful in assisting in the redispersion of abiraterone acetate from the carrier and into an aqueous suspension once the composition is ingested. Such excipients may be chosen based on their hydrophilic character, their wettability or their hygroscopicity, or their characteristics as disintegrants. The excipients may also include fillers or bulking agents that may assist in the further compounding of formulating of the pharmaceutical composition into a suitable dosage form.

Suitable excipients include hydroxyl-containing, hydrophilic, relatively low molecular weight (e.g., less than 50,000) compounds such as sugars, including monosaccharides, disaccharides, trisaccharides, sucrose, raffinose, lactose, mannitol, sorbitol, trehalose, glycerol, dextrose, fructose, pentoses, hexoses, xylitol and mixtures thereof.

Excipients may be useful as protectants in drying processes, such as cryoprotectants in a lyophilization process or as additives in a spray drying process or an evaporation process, preventing or substantially reducing particle fusion, combination and suspension degradation during drying, and assisting in the resuspension of particles from a dried state to form a suspension of the particles. Dry small particles containing the drug can be produced for example as a lyophilizate which is a solid produced from a cooled dispersion of particles by the process of freezing the aqueous carrier to a solid comprising a dispersion in ice and then removing the water by subliming the ice under reduced pressure. The excipients can also reduce or depress the freezing point of aqueous compositions in which 10 they are dissolved or partially dissolved.

The excipients can be added in amounts from 0.1% to about 60% w/w or more depending on the intended use. For example, in amounts of about 1%, 2%, 5%, 10%, 15%, 20%, 25%, 30%, 35%, 40%, 45%, 50% or 55% w/w.

In a particular embodiment, the pharmaceutical carrier comprises carbohydrates and/or sugar or sugar alcohol such as monosaccharides, disaccharides, trisaccharides, sucrose, raffinose, lactose, mannitol, sorbitol, trehalose, glycerol, dextrose, fructose, a pentose, a hexose, xylitol, and mixtures thereof. These excipients may be employed in amounts of about 5% to about 40%, preferably about 10% to about 30% of the total weight of the 20 pharmaceutical composition.

The pharmaceutical composition described above may be in a liquid, semi-solid or solid particulate form and employed as such as a finished dosage form suitable for administration to a mammalian subject. However, in many cases it will be more convenient to present the pharmaceutical composition in a dosage form together with additional pharmaceutical adjuvants. Accordingly, the pharmaceutical composition can be further compounded or formulated with other pharmaceutically acceptable adjuvants to produce a dosage form. For example, the composition may be dried, and granulated with said adjuvants before being filled into capsules or sachets, suspended in a syrup, or compacted to folui a tablet. The selection of pharmaceutical adjuvants can be made having regard to the nature of the intended dosage form.

The dosage form may be presented in the form of solid dosage forms including tablets, beads, capsules, grains, pills, granulates, granules, powder, pellets, sachets, lozenges, troches and the like. In a powdered form dispersible in a beverage, or suspended or dissolved in a liquid oil form.

Suitable pharmaceutical adjuvants include one or more materials selected from the group of fillers, binders, lubricants, disintegrants, sweeteners, colours, glidants, surfactants, and the like.

Suitable fillers may include one or more of microcrystalline cellulose, silicified microcrystalline cellulose, mannitol, calcium phosphate, calcium sulphate, kaolin, dry starch, powdered sugar, and the like, lactose (e.g. spray-dried lactose, a-lactose, b-lactose, Tabletose(R), various grades of Pharmatose(R), Microtose(R) or Fast-Floe(R)), microcrystalline cellulose (various grades of Avicel(R), Elcema(R), Vivacel(R), Ming Tai(R) or Solka-Floc(R)), hydroxypropylcellulose, L-hydroxypropylcellulose (low substituted), hydroxypropyl methylcellulose (HPMC) (e.g., Methocel E, F and K, Metolose SH of Shin-Etsu, Ltd, such as, e.g. the 4,000 cps grades of Methocel E and Metolose 60 SH, the 4,000 cps grades of Methocel F and Metolose 65 SH, the 4,000, 15,000 and 100,000 cps grades of Methocel K; and the 4,000, 15,000, 39,000 and 100,000 grades of Metolose 90 SH), methylcellulose polymers (such as, e.g., Methocel A, Methocel A4C, Methocel A15C, Methocel A4M), hydroxyethylcellulose, sodium carboxymethylcellulose, carboxymethylene, carboxymethylhydroxyethylcellulose and other cellulose derivatives, sucrose, agarose, sorbitol, mannitol, dextrins, maltodextrins, starches or modified starches (including potato starch, maize starch and rice starch), calcium phosphate (e.g., basic calcium phosphate, calcium hydrogen phosphate, dicalcium phosphate hydrate), calcium sulphate, calcium carbonate, sodium alginate, collagen and the like.

Suitable binders may include one or more of povidone, starch, stearic acid, gums, hydroxypropylmethyl cellulose, acacia, alginic acid, agar, calcium carrageenan, sodium carboxymethylcellulose, microcrystalline cellulose, dextrin, ethylcellulose, gelatin, liquid glucose, guar gum, hydroxypropyl methylcellulose, methylcellulose, pectin, PEG, povidone, pregelatinized starch and the like.

Other adjuvants in a dosage form according to the invention may be antioxidants like e.g. ascorbic acid, ascorbyl palmitate, butylated hydroxyanisole (BHA), butylated hydroxytoluene (BHT), hypophosphorous acid, monothioglycerol, potassium metabisulfite, propyl gallate, sodium formaldehylde sulfoxylate, sodium metabisulfite, sodium thio sulfate, tocopherol, tocopherol acetate, tocopherol hemisuccinate, TPGS or other tocopherol derivatives. The carrier composition may also contain e.g., stabilising agents. The concentration of an antioxidant and/or a stabilizing agent in the carrier composition is normally from about 0.1 w/w to about 5% w/w.

Suitable lubricants may include one or more of magnesium stearate, zinc stearate, calcium stearate, stearic acid, sodium stearyl fumarate, hydrogenated vegetable oil, glyceryl behenate, talc, magnesium stearate, calcium stearate, stearic acid, colloidal silicon dioxide, magnesium carbonate, magnesium oxide, calcium silicate, microcrystalline cellulose, starches, mineral oil, waxes, glyceryl behenate, polyethylene glycol, sodium benzoate, sodium acetate, sodium chloride, sodium laurylsulfate, sodium stearyl fumarate, and hydrogenated vegetable oils. Preferably, the lubricant is magnesium stearate or talc, more particularly magnesium stearate and talc in combination.

Suitable disintegrants may include one or more of starch, croscarmellose sodium, crospovidone, sodium starch glycolate, alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel(R) and Explotab(R)), starches, clays, celluloses, alginates, gums, cross-linked polymers (such as cross-linked polyvinylpyrrolidone and cross-linked sodium carboxymethylcellulose), sodium starch glycolate, low-substituted hydroxypropyl cellulose, and soy polysaccharides. Preferably, the disintegrant is a modified cellulose gum such as e.g. cross-linked sodium carboxymethylcellulose.

Suitable glidants may include one or more of colloidal silicon dioxide, talc and the like.

Colouring agents may be selected from any FDA approved colours for oral use.

The total amount of additional excipients that can be added ranges from about 0.1% to about 50%, preferably from 1% to about 30%, and more preferably from about 2% to about 30% based on the total weight of the pharmaceutical composition.

In another aspect of the invention there is provided a process of forming a pharmaceutical composition comprising abiraterone acetate dissolved or dispersed in a pharmaceutically acceptable carrier as hereinabove described, said process comprising the step of dispersing abiraterone acetate in a carrier containing one or more lipid excipients or surfactants and/or co-surfacants, and optionally one or more pharmaceutically acceptable excipients.

Such a process comprises contacting abiraterone acetate with the carrier and any of the other optional excipients for a time and under conditions sufficient to provide a dispersion or dissolution of abiraterone acetate in the carrier material. The additional excipients can be added before, during, or after dispersion or dissolution of abiraterone acetate.

Dissolution or dispersion of the drug substance in the carrier material can be performed by a process of homogenisation. The homogenization step may assist in particle size reduction, which can also give rise to improved bioavailability. Homogenisation techniques are well known in the art and do not merit a detailed discussion here. Examples of such a process are provided in the examples below.

The pharmaceutical composition formed by dissolution or dispersion of abiraterone acetate in a pharmaceutically acceptable carrier may be used as finished dosage forms, or it may be filled into capsules, or it can be dried to form a powder using conventional drying techniques such as spray-drying onto beads or non-pareils, freezing, lyophilisation or any other suitable technique known in the art.

The dried pharmaceutical compositions of the present invention can be used as finished dosage forms for administration to a patient, or they can be further blended, compounded or formulated with pharmaceutical adjuvants as hereinabove described, in preparation for tabletting.

Finished dosage forms as tablets may be prepared utilizing conventional tabletting techniques. A general method of manufacture involves blending the dried pharmaceutical composition obtained from the drying step described hereinabove with a water-soluble or amphiphilic diluent, hydrophilic binder and optionally a portion of a disintegrant. Any other ingredients, such as lubricants, (e.g. magnesium stearate) and additional disintegrants may be added to the granules and mixed. This mixture is then compressed into a suitable size and shape using conventional tabletting machines such as a rotary tablet press.

If capsules are to be prepared, they may also be formed by utilizing conventional methods. A general method of manufacture involves blending the pharmaceutical composition described hereinabove with a water-soluble or amphiphilic diluent, hydrophilic binder and optionally a portion of a disintegrant. Any other ingredients, such as lubricants, (e.g. magnesium stearate) and additional disintegrants, may be added and mixed. The resulting mixture may then be filled into a suitable size hard-shell gelatin capsule using conventional capsule-filling machines.

When administered orally, abiraterone acetate does exhibit a first pass hepatotoxic effect. This can influence the dosage strength administered to a patient, particularly if the patient exhibits hepatic impairment. Accordingly, the dosage form of the present invention may be in the form of a tablet, film, wafer or the like that is adapted for buccal/sub-lingual administration.

There now follows a series of examples that serve to illustrate the invention.

### EXAMPLE 1

### General Manufacturing Process

A nanosuspension is formed according to the following procedure:

10% w/w abiraterone acetate, 1.5% w/w vitamin E TPGS and water qsp 100% w/w are mixed together with stirring to form a suspension. The suspension is passed through an Avestin C50 High Pressure Homogenizer (HPH) while its temperature is maintained at 20.° C. First the suspension is processed at a homogenizing pressure of 500 bars for ten minutes and then at 1500 bars. After 90 minutes of high pressure homogenization, the particle size of the nanosuspension is measured by LD: D50=0.3 micron, D90=0.9 micron and D99=0.6 micron. The mean particle size, measured by PCS (Photon Correlation Spectroscopy), is 330 nm with a polydispersity index (PI) of 0.2.

A powder blend is formed according to the following procedure:

Drying of the resulting fine suspension is conducted in a top spray fluid bed dryer. First, the fine suspension is mixed with maltodextrin and sodium lauryl sulfate. Then the mixture is sprayed onto lactose monohydrate.

The particle size distribution of the resuspended dried nanosuspension is then measured by LD (Laser Diffraction) and PCS (Photon Correlation Spectroscopy). The Particle Size Distribution (PSD) obtained by LD is as follow: D50=0.4 micron, D90=0.6 micron and D99=0.8 micron. The mean particle size measured by PCS is 438 nm with a PI of 0.4. The blend obtained after drying in the fluid bed dryer has the following composition:

| | |
|---|---|
| Abiraterone acetate | 28.6% |
| Lactose monohydrate | 42.9% |
| Maltodextrin | 20% |
| Vit E TPGS | 4.3% |
| Sodium lauryl sulfate | 4.3% |

Final blend for tabletting is prepared by mixing in a bin mixer the drier granules with additional ingredients and has the following composition

| | |
|---|---|
| Fluid bed dryer blend | 57.3% |
| Pearlitol DC | 24.6% |
| AcDiSol | 16.4% |
| Aerosil 200 | 0.8% |
| Magnesium stearate | 0.9% |

Tablets are pressed on a tablet press Korsch XL 100 at the nominal weight of 610 mg for a theoretical content of 100 mg of abiraterone acetate or at a nominal weight of 305 mg for a theoretical content of 50 mg of abiraterone acetate. High weight tablets are for oral administration whereas low weight tablets are adapted for sublingual delivery of the drug.

### EXAMPLE 2

### General Manufacturing Process

A nanosuspension is formed according to the following procedure:

10% w/w abiraterone acetate, 0.5% w/w vitamin E TPGS, 2.2% w/w Lipoid S75 and water qsp 100% w/w are mixed together with stirring to form a suspension. The suspension is passed through an Avestin C50 High Pressure Homogenizer (HPH) while its temperature is maintained at 20° C. First the suspension is processed at a homogenizing pressure of 500 bars for ten minutes and then at 1500 bars. After 120 minutes of high pressure homogenization, the particle size of the nanosuspension is measured by LD: D50=0.5 micron, D90=0.7 micron and D99=0.8 micron. The mean particle size, measured by PCS (Photon Correlation Spectroscopy), is 363 nm with a polydispersity index (PI) of 0.3.

A powder blend is formed according to the following procedure:

Drying of the resulting fine suspension is conducted in a top spray fluid bed dryer (GPCG1). First, the fine suspension is mixed with maltodextrin and sodium lauryl sulfate. Then the mixture is sprayed onto lactose monohydrate.

The particle size distribution of the resuspended dried nanosuspension is then measured by LD (Laser Diffraction) and PCS (Photon Correlation Spectroscopy). The Particle Size Distribution (PSD) obtained by LD is as follow: D50=0.6 micron, D90=0.8 micron and D99=0.9 micron. The mean particle size measured by PCS is 462 nm with a PI of 0.3.

The blend obtained after drying in the fluid bed dryer has the following composition

| | |
|---|---|
| Abiraterone acetate | 25% |
| Lactose monohydrate | 37.5% |
| Maltodextrin | 17.5% |
| Vit E TPGS | 3.75% |
| Sodium lauryl sulfate | 3.75% |
| LIPOID S75 | 12.5% |

Final blend for tabletting is prepared by mixing in a bin mixer the dry granules with additional ingredients and has the following composition.

| | |
|---|---|
| Fluid bed dryer blend | 60.6% |
| Pearlitol DC | 22.7% |
| AcDiSol | 15.0% |
| Aerosil 200 | 0.8% |
| Magnesium stearate | 0.9% |

Tablets are pressed on a tablet press Korsch XL 100 at the nominal weight of 660 mg for a theoritical content of 100 mg of abiraterone acetate or at a nominal weight of 330 mg for a theoritical content of 50 mg of abiraterone acetate.

High weight tablets are for oral administration whereas low weight tablets are adapted for sublingual delivery of the drug.

### EXAMPLE 3

Under agitation Abiraterone acetate is dispersed in molten mixture of Gelucire 50/13 and 10 Lipoid S75. The molten mixture is then processed through a high pressure homogenizer.

After this treatment the hot mixture is then poured into hard shell capsule at a theoretical weight of 833 mg corresponding to 250 mg of abiraterone acetate. The content of the capsule has the following composition.

| | |
|---|---|
| Abiraterone acetate | 30% |
| Gelucire 50/13 | 60% |
| Lipoid S 75 | 10% |

Molten preparation is poured in a capsule size 00el.

1. A pharmaceutical composition comprising abiraterone acetate dissolved or dispersed in a pharmaceutically acceptable carrier, wherein the pharmaceutically acceptable carrier comprises one or more lipid excipients, selected from the group consisting of fatty acid esters, phospholipids, phosphatidyl compounds, glycosylceramides, fatty acids, nonionic surfactants, vitamin E tocopheryl succinate polyethylene glycol, glycerides, derivatives thereof, and mixtures thereof.

2. A pharmaceutical composition according to claim 1 further comprising one or more surfactants.

3. A pharmaceutical composition according to claim 1 wherein the lipid excipient is a polyethylene glycol glyceride.

4. A pharmaceutical composition according to claim 3 wherein the polyethylene glycol glyceride is lauroyl macrogol-32 glycerides.

5. A pharmaceutical composition according to claim 1, further comprising one or more pharmaceutically acceptable excipients.

6. A pharmaceutical composition comprising abiraterone acetate dissolved or dispersed in a pharmaceutically acceptable carrier, which composition when administered in a 250 mg single dose orally to a mammalian subject in the fasted state exhibits improved bioavailability compared to a 250 mg orally administered single dosage form of the drug product that is commercially marketed under the trade name ZYTIGA®.

7. The method of claim 10, wherein the composition, when administered to a mammalian subject in the fasted state, is bioequivalent to the same dosage form administered to a mammalian subject in a fed state.

8. The method of claim 10, wherein when the composition is administered to a mammalian subject orally in fasting conditions at a single dose of 1000 mg, the composition exhibits a pharmacokinetic profile characterised by an AUC₀₋₂₄ greater than 421 nM/1.ml.

9. The method of claim 10, wherein when the composition is administered to a mammalian subject orally in fasting conditions at a single dose of 1000 mg, the composition exhibits a pharmacokinetic profile characterised by a Cmax of about 70 ng/ml.

10. A method of reducing or eliminating the difference between one or more bioequivalence parameters measured after oral administration of a pharmaceutical composition to a mammalian subject in a fasted state compared with administration of the same composition to a mammalian subject in a fed state, the method comprising the step of administering to a mammalian subject a pharmaceutical composition according to claim 1.

11. (canceled)

12. A method of treating cancer in a patient in need thereof comprising administering to the patient a dosage form according to claim 1.

13. (canceled)

14. (canceled)

15. The method of claim 12, wherein the cancer is prostate cancer.

16. (canceled)
